(19) United States
(12) Patent Application Publication
(10) Pub. No.: US 2004/0072217 A1
(43) Pub. Date: Apr. 15, 2004

(54) Methods of analysis of linkage disequilibrium

(75) Inventor: Giulia Kennedy, San Francisco, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/463,991

(22) Filed: Jun. 17, 2003

Related U.S. Application Data
(63) Continuation-in-part of application No. 10/264,945, filed on Oct. 4, 2002.
(60) Provisional application No. 60/389,701, filed on Jun. 17, 2002.
(60) Provisional application No. 60/389,746, filed on Jun. 17, 2002.

(51) Int. Cl. C12Q 1/68; C12P 19/34

(52) U.S. Cl. 435/6; 435/91.2

(57) ABSTRACT

Methods and kits for analyzing a collection of target sequences in a nucleic acid sample are provided. A sample is amplified under conditions that enrich for a subset of fragments that includes a collection of target sequences. Methods are also provided for analysis of the above sample by hybridization to an array, which may be specifically designed to interrogate the collection of target sequences for particular characteristics, such as, for example, the presence or absence of one or more polymorphisms. Methods of estimating the extent of linkage disequilibrium in a region or population by determination of the ancestral and non-ancestral alleles are also provided.

### RELATED APPLICATIONS

The present application claims priority to U.S. Provisional Application Nos. 60/389,701 and 60/389,746 filed Jun. 17, 2002 and is a continuation-in-part of U.S. application Ser. No. 10/264,945 filed Oct. 4, 2002 the disclosures of which are each incorporated herein by reference in their entireties.

### FIELD OF THE INVENTION

The invention relates to methods for enrichment and amplification of sequences from a nucleic acid sample and highly parallel methods of determining the genotypes of SNPs. In many embodiments a generic method of complexity reduction is combined with an array of probes to a collection of SNPs to determine genotype. In one embodiment, the invention relates to determining regions of low or high linkage disequilibrium across the whole genome. In another embodiment the invention relates to identification of ancestral alleles of human polymorphisms. The methods may be used to identify human chromosomal regions of low linkage disequilibrium and to determine haplotype maps. The present invention relates to the fields of molecular biology and genetics.

### SUMMARY OF THE INVENTION

In one embodiment a method for identifying the ancestral allele of a human single nucleotide polymorphism is provided. Genomic DNA samples from at least two higher primate species are amplified by a method to reduce complexity, for example amplification with a single primer, and hybridized to a nucleic acid array comprising allele specific probes to at least 5,000 human SNPs. Hybridization patterns for the primates are analyzed to identify at least one human SNP that is homozygous for the same allele in both of the higher primate species. The allele present in both higher primate species is assigned as the ancestral allele state of that human SNP.

In another embodiment the extent of linkage disequilibrium in the chromosomal region near at least one human SNP is estimated by determining the ancestral allele for a plurality of human SNPs, identifying at least one human SNP allele that is the ancestral allele; and predicting low linkage disequilibrium across the chromosomal region near the SNP allele that is the ancestral allele or predicting low linkage disequilibrium across the chromosomal region near a non-ancestral allele. The non-ancestral allele is any allele that is not the ancestral allele. The higher primate species may be, for example, chimpanzee, gorilla or orangutan. The region may include the region 1, 10, 100 or 200 kb from the SNP in either direction.

In another embodiment the extent of linkage disequilibrium in the chromosomal region near a plurality of human SNPs is estimated by determining the ancestral allele for a plurality of human SNPs according to the method of claim, identifying the ancestral and non-ancestral alleles for each human SNP and predicting low linkage disequilibrium across the chromosomal region near each ancestral allele and high linkage disequilibrium across the chromosomal region near each non-ancestral allele.

In another embodiment a pattern of regions of high linkage disequilibrium across a chromosome is established by identifying at least one non-ancestral allele in a chromosome of an individual, determining the ancestral state of the human SNP in a population of individuals, identifying at least one human SNP that is found to be non-ancestral in a population of individuals with a frequency greater than 0.3, predicting regions of high linkage disequilibrium near a frequent non-ancestral allele on a chromosome of the population; and establishing a pattern of regions of high linkage disequilibrium across a chromosome. In another embodiment a pattern of regions of high linkage disequilibrium is established across a plurality of chromosomes by establishing a pattern of regions of high linkage disequilibrium across one chromosome and repeating for at least one other SNP located on a second chromosome.

In another embodiment a linkage disequilibrium map is established across human chromosomes by identifying at least one non-ancestral allele of a first human SNP, identifying chromosomal regions localized less than 200 kilobases from the at least one non-ancestral allele, identifying at least one other human SNP within this region; grouping the first SNP and the SNP or SNPs identified into blocks, and predicting high linkage disequilibrium between SNPs within these blocks. This may be used to establish a haplotype map. In another embodiment a computer and computer code are used to estimate haplotype diversity within blocks of SNPs and may be used to establish a haplotype map.

In another embodiment a haplotype map across a human chromosome is established by identifying human SNPs that are non-ancestral, identifying chromosomal regions localized less than 200 kb from the non-ancestral allele, identifying human SNPs within these regions, grouping the SNPs into blocks of high linkage disequilibrium, estimating haplotype diversity within these blocks via an haplotype estimation software and establishing an haplotype map.

In another embodiment the haplotype map that is established is used to search for complex disease genes.

In another embodiment the extent of linkage disequilibrium in the human genome is estimated by determining the allele frequencies for a first plurality of SNPs in a population, determining the ancestral allele for a second plurality of SNPs contained in the first plurality of SNPs, comparing the allele frequency of a SNP to the frequency of the ancestral allele for that SNP for a plurality of SNPs from the second plurality of SNPs in the population sample to generate a correlation coefficient for the population; determining high frequency for an ancestral allele if the correlation coefficient is greater than 0.8, identifying a chromosomal region nearby a frequent ancestral allele; and inferring low linkage disequilibrium across the region.

Populations that may be compared include any distinct populations, for example, geographically distinct human populations. The methods may be used to determine which population is more ancient.

In another embodiment at least one ancestry informative marker is identified by determining the allele frequency for each of a plurality of SNPs in each of two populations, calculating an F_{ST} value for at least one SNP in the plurality of SNPs and identifying at least one SNP whose F_{ST} value is greater than 0.3 or greater than 0.4.

In another embodiment a haplotype is identified in a region of high linkage disequilibrium in a first population of individuals, wherein a haplotype comprises at least two linked SNP alleles, by identifying a non-ancestral allele of a first SNP in the first population wherein the non-ancestral allele is not present in a second population, genotyping at least one additional SNP that is within 100 kb of the first SNP; and determining which allele of the additional SNP is linked to the non-ancestral allele of the first SNP.

### BACKGROUND

In recent years, there has been a great interest by human geneticist to map the entire human genome for the multiple genes involved in common complex disorders. With the increasing number of single nucleotide polymorphisms, such as those identified by the SNP Consortium and the novel methods of genotyping, association studies between DNA variants and disease will increase. Because of the limitations of other linkage methodologies, linkage disequilibrium mapping has become the strategy of choice to map complex diseases through the whole genome. Due to the wide ranging applications of SNPs there is still a need for the development of robust, flexible, cost-effective technology platforms that allow for scoring genotypes in large numbers of samples.

### DETAILED DESCRIPTION

A. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach ” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/USO1/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098.

Nucleic acid arrays are described in many of the above patents, but the same techniques are applied to polypeptide arrays. Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248, 6,309,822 and 6,344,316. Genotyping and uses therefore are shown in U.S. Pat. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5, 413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; 6,225,625, and 6,344,316 in U.S. Patent application No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Patent applications 10/063,559, 60/349,546, 60/376,003, 60/394,574, 60/403,381.

B. Definitions

An individual is not limited to a human being, but may also include other organisms including but not limited to mammals, plants, bacteria or cells derived from any of the above.

Nucleic acids according to the present invention may include any polymer or oligomer of pyrimidine and purine bases, preferably cytosine (C), thymine (T), and uracil (U), and adenine (A) and guanine (G), respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The analogs are those molecules having some structural features in common with a naturally occurring nucleoside or nucleotide such that when incorporated in a nucleic acid or oligonucleotide sequence, they allow hybridization with a naturally occurring nucleic acid sequence The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An oligonucleotide or polynucleotide is a single-stranded nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA) in which the constituent bases are joined by peptide bonds rather than phosphodiester linkages. (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide”, “nucleic acid” and “oligonucleotide” are used interchangeably in this application.

The term fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

A number of methods disclosed herein require the use of restriction enzymes to fragment the nucleic acid sample. In general, a restriction enzyme recognizes a specific nucleotide sequence of four to eight nucleotides and cuts the DNA at a site within or a specific distance from the recognition sequence. For example, the restriction enzyme EcoRI recognizes the sequence GAATTC and will cut a DNA molecule between the G and the first A. The length of the recognition sequence is roughly proportional to the frequency of occurrence of the site in the genome. A simplistic theoretical estimate is that a six base pair recognition sequence will occur once in every 4096 (4⁶) base pairs while a four base pair recognition sequence will occur once every 256 (4⁴) base pairs. In silico digestions of sequences from the Human Genome Project show that the actual occurrences may be even more infrequent for some enzymes and more frequent for others, for example, PstI cuts the human genome more often than would be predicted by this simplistic theory while SalI and XhoI cut the human genome less frequently than predicted. Because the restriction sites are rare, the appearance of shorter restriction fragments, for example those less than 1000 base pairs, is much less frequent than the appearance of longer fragments. Many different restriction enzymes are known and appropriate restriction enzymes can be selected for a desired result. (For a description of many restriction enzymes see, New England BioLabs Catalog (Beverly, Mass.) which is herein incorporated by reference in its entirety for all purposes).

Information about the sequence of a region may be combined with information about the sequence specificity of a particular restriction enzyme to predict the size, distribution and sequence of fragments that will result when a particular region of a genome is digested with that enzyme. In silico digestion is a computer aided simulation of enzymatic digests accomplished by searching a sequence for restriction sites. In silico digestion provides for the use of a computer or computer system to model enzymatic reactions in order to determine experimental conditions before conducting any actual experiments. An example of an experiment would be to model digestion of the human genome with specific restriction enzymes to predict the sizes and sequences of the resulting restriction fragments.

Adaptor sequences or adaptors are generally oligonucleotides of at least 5, 10, or 15 bases and preferably no more than 50 or 60 bases in length, however, they may be even longer, up to 100 or 200 bases. Adaptor sequences may be synthesized using any methods known to those of skill in the art. For the purposes of this invention they may, as options, comprise templates for PCR primers, restriction sites, tags and promoters. The adaptor may be partially, entirely or substantially double stranded. The adaptor may be phosphorylated or unphosphorylated on one or both strands. Modified nucleotides, for example, phosphorothioates, may also be incorporated into one or both strands of an adaptor.

Adaptors are particularly useful in some embodiments of the methods if they comprise a substantially double stranded region and short single stranded regions which are complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible.

In some embodiments the same adaptor sequence is ligated to both ends of a fragment. Digestion of a nucleic acid sample with a single enzyme may generate similar or identical overhanging or sticky ends on either end of the fragment. For example if a nucleic acid sample is digested with EcoRI both strands of the DNA will have at their 5′ ends a single stranded region, or overhang, of 5′-AATT-3′. A single adaptor sequence that has a complementary overhang of 5′-AATT-3′ can be ligated to each end of the fragment.

A single adaptor can also be ligated to both ends of a fragment resulting from digestion with two different enzymes. For example, if the method of digestion generates blunt ended fragments, the same adaptor sequence can be ligated to both ends. Alternatively some pairs of enzymes leave identical overhanging sequences. For example, BglII recognizes the sequence 5′-AGATCT-3′, cutting after the first A, and BamHI recognizes the sequence 5′-GGATCC-3′, cutting after the first G; both leave an overhang of 5′-GATC-3′. A single adaptor with an overhang of 5′-GATC-3′ may be ligated to both digestion products.

When a single adaptor sequence is ligated to both ends of a fragment the ends of a single fragment may be complementary resulting in the potential formation of hairpin structures. Formation of a base pairing interaction between the 5′ and 3′ ends of a fragment can inhibit amplification during PCR resulting in lowered overall yield. This effect will be more pronounced with smaller fragments than with larger fragments because the probability that the ends will hybridize is higher for smaller fragments than for larger fragments.

Digestion with two or more enzymes c an be used to selectively ligate separate adapters to either end of a restriction fragment. For example, if a fragment is the result of digestion with EcoRI at one end and BamHI at the other end, the overhangs will be 5′-AATT-3′ and 5′-GATC-3′, respectively. An adaptor with an overhang of AATT will be preferentially ligated to one end while an adaptor with an overhang of GATC will be preferentially ligated to the second end.

Methods of ligation will be known to those of skill in the art and are described, for example in Sambrook et at. and the New England BioLabs catalog both of which are incorporated herein by reference in their entireties. Methods include using T4 DNA Ligase which catalyzes the formation of a phosphodiester bond between juxtaposed 5′ phosphate and 3′ hydroxyl termini in duplex DNA or RNA with blunt or sticky ends; Taq DNA ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′ to 5′ phosphodiester bond, substrates include single-stranded RNA and DNA as well as dinucleoside pyrophosphates; or any other substrates described in the art.

“Genome” designates or denotes the complete, single-copy set of genetic instructions for an organism as coded into the DNA of the organism. A genome may be multi-chromosomal such that the DNA is cellularly distributed among a plurality of individual chromosomes. For example, in human there are 22 pairs of chromosomes plus a gender associated XX or XY pair.

An allele refers to one specific form of a gene within a cell or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

At each autosomal specific chromosomal location or “locus” an individual possesses two alleles, one inherited from the father and one from the mother. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles, each occurring at frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic or biallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Single nucleotide polymorphisms may be functional or non-functional. Functional polymorphisms affect gene regulation or protein sequence whereas non-functional polymorphisms do not. Depending on the site of the polymorphism and importance of the change, functional polymorphisms can also cause, or contribute to diseases.

SNPs can occur at different locations of the gene and may affect its function For instance: Polymorphisms in promoter and enhancer regions can affect gene function by modulating transcription, particularly if they are situated at recognition sites for DNA binding proteins. Polymorphisms in the 5′ untranslated region of genes can affect the efficiency with which proteins are translated. Polymorphisms in the protein-coding region of genes can alter the amino acid sequence and thereby alter gene function. Polymorphisms in the 3′ untranslated region of gene can affect gene function by altering the secondary structure of RNA and efficiency of translation or by affecting motifs in the RNA that bind proteins which regulate RNA degradation. Polymorphisms within introns can affect gene function by affecting RNA splicing.

The term genotyping or genotype refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Each allele may be present at a different frequency in a given population, for example 30% of the chromosomes in a population may carry the A allele and 70% the C allele. The frequency of the A allele would be 30% and the frequency of the C allele would be 70% in that population. Those individuals who have two copies of the C allele are homozygous for the C allele and the genotype is CC, those individuals who have two copies of the A allele are homozygous for the A allele and the genotype is AA, and those individuals who have one copy of each allele are heterozygous and the genotype is AC. The array may be designed to distinguish between each of these three possible outcomes. A polymorphic location may have two or more possible alleles and the array may be designed to distinguish between all possible combinations.

Normal cells that are heterozygous at one or more loci may give rise to tumor cells that are homozygous at those loci. This loss of heterozygosity may result from structural deletion of normal genes or loss of the chromosome carrying the normal gene, mitotic recombination between normal and mutant genes, followed by formation of daughter cells homozygous for deleted or inactivated (mutant) genes; or loss of the chromosome with the normal gene and duplication of the chromosome with the deleted or inactivated (mutant) gene.

Single nucleotide polymorphisms (SNPs) are positions at which two alternative bases occur at appreciable frequency (>1%) in a given population. SNPs are the most common type of human genetic variation. A polymorphic site is frequently preceded by and followed by highly conserved sequences (e.g., sequences that vary in less than 1/100 or 1/1000 members of the populations).

A SNP may arise due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

The “ancestral allele” is the allele that arose earlier in the evolution of the organism. Ancestral alleles for humans are frequently identified as the allele present in lower primates. Non-ancestral alleles are alleles that are different from the ancestral allele. Different non-ancestral alleles may be present in different populations. For example, if the ancestral allele for a SNP is A, one population may have a non-ancestral allele that is G, a second population may have a non-ancestral allele that is C and a third population may be homozygous for the ancestral allele. Some SNPs in some populations may have 2 or 3 non-ancestral alleles represented.

A phenotype refers to any visible, detectable or otherwise measurable property of an organism such as symptoms of, or susceptibility to a disease for example.

A haplotype is a combination of multiple alleles or genetic markers at neighboring loci on a single chromosome of a given individual. Estimation of haplotype frequencies from genotype data can be accomplished through statistical algorithms such as the expectation-maximation algorithm or E-M algorithm (Excoffier and Slatkin. (1995), Molecular Biology of Evolution, 12:921-927). The E-M algorithm uses haplotype frequencies from unambiguous individuals to project and infer haplotypes from the ambiguous individuals. The E-M algorithm first computes expected genotype probabilities based on haplotype frequency estimates provided by genotype data from individuals with complete information and projected frequency information for individuals that have ambiguous genotypes. This is the ‘expectation’ step. Once estimates of the frequencies are obtained, the probability of each possible pair of haplotypes for each individual's genotype configuration is computed. These probabilities provide information about how compatible the estimated haplotype frequencies are with the genotype data. This step is the ‘maximization’ step. These two steps are pursued in sequence until the estimates converge (i.e., do not change with subsequent expectation and maximization calculations). Examples of haplotype estimation software include Arlequin (Excoffier and Slatkin (1995)), Haplo (Hawley and Kidd (1995), J. Hered., 86:409-411) and Genehunter (Krugylak et al. (1996), Am. J. Hum. Genet. 58:1347-1363).

A “haplotype map” refers to a combination of biallelic markers or biallelic SNPs found in a given individual and which may be associated with a phenotype. For example, a haplotype map can be an individual's genotype for multiple loci or SNPs on a single chromosome.

The term linkage disequilibrium (LD) refers to a population association among alleles at two or more loci. It is a measure of co-segregation of alleles in a population. Linkage disequilibrium or allelic association is the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

A marker in linkage disequilibrium can be particularly useful in detecting susceptibility to disease (or other phenotype). The marker may or may not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

The term allele frequency corresponds to the fraction of the number of individuals with a given allele over the total number of alleles in the population tested.

A population is a group (usually a large group) of individuals. Human population samples corresponds to samples chosen from a population defined by, for example, ethnicity (population of origin) and geography. For example population sample could be chosen from different ethnic group such as: African, African-American, Caucasian, Asian, Asian-American, Chinese, Chinese-American, and also depending on the geography: for example Chinese-American from Hawaii. Alternatively, human population samples can be chosen from an experimental population such as individuals in a diseased population or individuals that react in a particular manner when administered a drug and compared to a control population such as healthy individuals.

Wright's statistic (F_{ST}) is used to detect variation between samples. Based on the relative frequency of different alleles, the genetic distance between populations can be estimated. F_{ST} is a measure of the difference in allele frequencies between populations and therefore measures the overall divergence among populations. A F_{ST} value of 0 shows that a SNP has the same allele frequency between two populations. A F_{ST} value close to 1 indicates that a SNP has very large allele frequency differences between two populations. Thus, if the F_{ST} value is close to 1, the two populations studied show statistically significant genetic differences. SNPs with a high F_{ST} value may indicate past or present selective pressure in one population. A high F_{ST} value may be used to identify SNPs that are of interest for correlation with selective pressure, for example.

The term correlation coefficient is a measure of the strength of the linear relationship between two variables X and Y. A linear relationship is defined by the equation Y=aX+B wherein a is the slope of the line. A correlation coefficient is a number between −1 and 1. The maximum positive correlation is 1 and corresponds to a perfect linear relationship with a positive slope between the two variables. The maximum negative correlation is −1 and corresponds to a perfect linear relationship with a negative slope between the two variables. A 0 or near 0 correlation coefficient represents a complete independence between the two variables.

C. Preferential Amplification of a Subset of Fragments Containing Target Sequences

The present invention provides for novel methods of analysis of a nucleic acid sample, such as genomic DNA. The methods include: identification and selection of a collection of target sequences; amplification of a selected subset of fragments that comprises a collection of target sequences; and, analysis of a collection of target sequences. In many embodiments the target sequences comprise a SNP. In many embodiments a subset of fragments may be amplified by PCR wherein the subset of fragments that is amplified efficiently is dependent on the size of the fragments. In one embodiment fragmentation conditions and target sequences are selected so that the target sequences are present in the subset of fragments that are efficiently amplified by PCR. Those fragments that are efficiently amplified are enriched in the amplified sample and are present in amounts sufficient for hybridization analysis and detection using the methods disclosed. Many fragments will not be amplified efficiently enough for detection using the methods disclosed and these fragments are not enriched in the amplified sample.

In many embodiments the methods include the steps of: identifying a collection of target sequences that carry polymorphisms on fragments of a selected size range when the genome is digested with a selected enzyme or enzymes; fragmenting a nucleic acid sample by digestion with one or more restriction enzymes so that the target sequences are present on fragments that are within the selected size range; ligating one or more adaptors to the fragments; and amplifying the fragments so that a subset of the fragments, including fragments of the selected size range, are enriched in the amplified product. In some embodiments the amplified sample is exposed to an array which may be specifically designed and manufactured to interrogate one or more target sequences in a collection of target sequences. The array may be designed to genotype more than 5,000, more than 10,000 or more than 100,000 different poltmorphisms.

In some embodiments the selected size range is selected to be within the size range of fragments that can be efficiently amplified under a given set of amplification conditions. In many embodiments amplification is by PCR and the PCR conditions are standard PCR amplification conditions (see, for example, PCR primer A laboratory Manual, Cold Spring Harbor Lab Press, (1995) eds. C. Dieffenbach and G. Dveksler), under these conditions fragments that are of a predicted size range, generally less than 2 kb, will be amplified most efficiently.

Before hybridization to an array in many embodiments the genomic sample is amplified so that a subset of fragments is enriched in the amplified product relative to the remaining fragments. All fragments that have the adaptor sequence ligated to both ends are substrates for amplification but some will be amplified more efficiently than others. It is possible to predict which fragments will be amplified more efficiently and which fragments will be amplified less efficiently. Those fragments that are amplified more efficiently will be enriched in the amplified product relative to the fragments that are amplified less efficiently. Differing amplification conditions will result in different in enrichment of different subsets of fragments. Efficiency of amplification is related to the size of a fragment so different fragmentation conditions will also result in enrichment of different subsets of fragments. In some embodiments the fragments that will be amplified more efficiently are predicted and SNPs that are present on those fragments are selected for genotyping.

In some embodiments fragments that are about 2 kb and less are predicted to be efficiently amplified and therefore enriched in the amplified product. In another embodiment fragments that are between 1 and 4 kb are predicted to be efficiently amplified and enriched in the amplified product. In some embodiments 2, 20, 50, 100, or 1,000 samples may be fragmented and amplified under the same conditions in parallel and genotyped using a genotyping array that genotypes the same set of SNPs and resulting in a call rate of 90% or greater for the SNPs (meaning that for each sample at least 90% of the SNPs in the set are given a genotype). Using the disclosed methods a collection of SNPs may be reproducibly genotyped in a plurality of individuals.

SNPs that are predicted to be present on fragments that are enriched in the amplified sample may be selected for genotyping on the array or not based on secondary considerations such as performance in hybridization experiments, location in the genome, proximity to another target sequence in the collection, association with phenotype or disease or any other criteria that is known in the art. For example, if two SNPs are within 1 kb of one another it may be desirable to genotype only one of the SNPs. Additional selection criteria that may be used to select target sequences for a collection of target sequences also include, for example, clustering characteristics, whether or not a SNP is consistently present in a population, Mendelian inheritance characteristics, Hardy-Weinberg probability, and chromosomal map distribution. In one embodiment fragments that contain repetitive sequences, telomeric regions, centromeric regions and heterochromatin domains may be excluded. In one embodiment the SNPs are selected to provide an optimal representation of the genome. For example SNPs may be selected so that the distance between SNPs in the target collection is on average between 10, 50, 100, 200 or 300 and 50, 100, 200, 400, 600 or 800 kb. Inter-SNP distances may vary from chromosome to chromosome. In one embodiment more than 80% of the SNPs are less than about 200 kb from another SNP that is being genotyped. In another embodiment more than 80% of the SNPs are less than about 10, 50, 100, 150, 300 or 500 kb from another SNP being genotyped. In one embodiment SNPs that give errors across multiple families are not selected or are removed from the analysis. In another embodiment SNPs that give ambiguous results in multiple experiments are not selected or are removed from the analysis.

In many embodiments the methods employ the use of a computer system to assist in the identification of SNPs for genotyping. For many organisms, including yeast, mouse, human and a number of microbial species, a complete or complete draft of the genomic sequence is known and publicly available. Knowledge of the sequences present in a nucleic acid sample, such as a genome, allow prediction of the sizes and sequence content of fragments that will result when the genome is fragmented by a given method. The pool of predicted fragments may be analyzed to identify which fragments are within a selected size range, which fragments carry a polymorphism and which fragments have both characteristics. In some embodiments an array is then designed to genotype at least some of the polymorphisms. A nucleic acid sample may then be digested with the selected enzyme or enzyme and amplified under the selected amplification conditions, resulting in the amplification of the collection of target sequences. The amplified target sequences may then be analyzed by hybridization to the array. In some embodiments the amplified sequences may be further analyzed using any known method including sequencing, HPLC, hybridization analysis, cloning, labeling, etc.

In one embodiment a nucleic acid sample is digested with two or more enzymes that cut the DNA to produce compatible cohesive ends. A compatible cohesive end results when two enzymes cleave at different recognition sites but generate identical overhangs, for example, Xba I recognizes the sequence 5′-TCTAGA-3′ and AvrII recognizes 5′-CCTAGG-3′ but both result in an overhang that is 5′-CTAG-3′ as shown below:

XbaI: Recognition: Cleavage:

| | | |
|---|---|---|
| 5′-TCTAGA-3′ | 5′-T | CTAGA-3′ |
| 5′-AGATCT-3′ | 5′-AGATC | T-3′ |

AvrII: Recognition: Cleavage:

| | | |
|---|---|---|
| 5′-CCTAGG-3′ | 5′-C | CTAGG-3′ |
| 5′-GGATCC-3′ | 5′-GGATC | C-3′ |

Fragments that are cut with either XbaI or AvrII will have the same overhang or sticky end, 5′-CTAG-3′ and may be ligated to a single adapter sequence with an overhang that is 5′-CTAG-3′. Genomic DNA may be digested in one reaction with two or more enzymes or digested in separate digest reactions with one or more enzymes then mixed and ligated in a single reaction to the adapter sequence.

Different combinations of enzymes may be selected to produce an amplified sample of varying complexity. For example, digestion with XbaI followed by amplification results in enrichment of a subset of the genome that is approximately 43 Mb and digestion with AvrII results in enrichment of a similar level of complexity. The combination of XbaI and AvrII results in complexity that is approximately 80 Mb. The population of SNPs that are expected to be amplified is approximately the combination of SNPs that would be expected if either enzyme alone was used.

In some embodiments two or more enzymes that digest DNA under similar or identical buffer conditions are selected and the DNA is simultaneously digested with the two or more enzymes in a single reaction. If the enzymes produce the same overhang the DNA may then be ligated to the same adapter and amplified in a single reaction.

In one embodiment an enzyme that has a 4 base pair recognition site that is contained within the recognition site of a 6 base pair recognition site for a second restriction enzyme is used for digestion. For example, BfaI recognizes 5′-CTAG-3′ generating an overhang of 5′-CTAG-3′ which is identical to the overhang generated by XbaI. The BfaI recognition site is located within each XbaI site so digestion with BfaI will result in cleavage of XbaI sites as well as other sites in the genome that contain the 5′-CTAG-3′ sequence. The SNPs that are expected when the genome is digested with Xba I and amplified by the methods disclosed should also be amplified if the genome is digested with BfaI. Some SNPs that are present on fragments of, for example, 200 to 1000 in an XbaI digest may be on fragments that are smaller than 200 bp in a BfaI digest and would not be efficiently amplified. Those SNPs that might be less efficiently amplified in the BfaI digest may be predicted by in silico analysis.

In some embodiments a first set of SNPs that are present on fragments in a selected size range when a genome is digested with a first enzyme are identified and a second set of SNPs that are present on fragments in the same selected size range when the genome is digested with a second enzyme are identified and an array of probes is generated to genotype the alleles in the first and second set of SNPs. The array may be used to genotype only the first set of SNPs, only the second set of SNPs or both sets of SNPs simultaneously.

In some embodiments fractions are combined together to generate increased complexity. Increasing complexity target samples may be generated by, for example, adding various combinations of XbaI, EcoRI, BglII fractions, as well as fractions from other enzymes (BsrI, NcoI, HpaII, BspI, NdeI, HinP1). In one embodiment extremely high complexity samples (>300 Mb) are genotyped at >99% accuracy by increasing target DNA concentrations.

In another embodiment the SNPs present on fragments of a selected size range following fragmentation are selected as target sequences and an array is designed to interrogate at least some of the SNPs. For example, an array may be designed to genotype some of the SNPs that are present on fragments of 400 to 800 base pairs when human genomic DNA is digested with XbaI. If, for example, there are 15,000 SNPs that meet these criteria a subset of these SNP, for example, 10,000 may be selected for the array.

In one embodiment in silico prediction of the size of SNP containing fragments is combined with selection of a collection of target sequences to design genotyping assays and arrays for genotyping, see FIG. 4. In one embodiment target sequences are selected from fragments that are those in the size range of 400 to 800 base pairs, but other size ranges could also be used, for example, 100, 200, 500, 700, or 1,500 to 500, 700, 1,000, or 2,000 base pairs may also be useful size ranges.

In this embodiment an array may be designed to interrogate the SNPs that are predicted to be found in a size fraction resulting from digestion of the first nucleic acid sample with one or more particular restriction enzymes. For example, a computer may be used to search the sequence of a genome to identify all recognition sites for the restriction enzyme, EcoRI. The computer can then be used to predict the size of all restriction fragments resulting from an EcoRI digestion and to identify those fragments that contain a known or suspected SNP or polymorphism. The computer may then be used to identify the group of SNPs that are predicted to be found on fragments of, for example, 400-800 base pairs, when genomic DNA is digested with EcoRI. An array may then be designed to interrogate that subset of SNPs that are found on EcoRI fragments of 400-800 base pairs.

Arrays will preferably be designed to interrogate from 100, 500, 1000, 5000, 8000, 10,000, or 50,000 to 5,000, 10,000, 15,000, 30,000,100,000, 500,000 or 1,500,000 different SNPs. For example, an array may be designed to interrogate a collection of target sequences comprising a collection of SNPs predicted to be present on 400-800 base pair EcoRI fragments, a collection of SNPs predicted to be present on 400-800 base pair BglII fragments, a collection of SNPs predicted to be present on 400-800 base pair XbaI fragments, and a collection of SNPs predicted to be present on 400-800 base pair HindIII fragments. One or more amplified subsets of fragments may be pooled prior to hybridization to increase the complexity of the sample.

In some embodiments a single size selected amplification product is suitable for hybridization to many different arrays. For example, a single method of fragmentation and amplification that is suitable for hybridization to an array designed to interrogate SNPs contained on 400-800 base pair EcoRI would also be suitable for hybridization to an array designed to interrogate SNPs contained on 400-800 base pair BamHI fragments. This would introduce consistency and reproducibility to sample preparation methods.

In some embodiments SNPs present in a collection of target sequences are further characterized and an array is designed to interrogate a subset of these SNPs. SNPs may be selected for inclusion on an array based on a variety of characteristics, such as, for example, allelic frequency in a population, distribution in a genome, hybridization performance, genotyping performance, number of probes necessary for accurate genotyping, available linkage information, available mapping information, phenotypic characteristics or any other information about a SNP that makes it a better or worse candidate for analysis.

In many embodiments a selected collection of target sequences may be amplified reproducibly from different samples or from the same sample in different reactions. In one embodiment a plurality of samples are amplified in different reactions under similar conditions and each amplification reaction results in amplification of a similar collection of target sequences. Genomic samples from different individuals may be fragmented and amplified using a selected set of conditions and similar target sequences will be amplified from both samples. For example, if genomic DNA is isolated from 2 or more individuals, each sample is fragmented under similar conditions, amplified under similar conditions and hybridized to arrays designed to interrogate the same collection of target sequences, more than 50%, more than 75% or more than 90% of the same target sequences are detected in the samples.

A given target sequence may be present in different allelic forms in a cell, a sample, an individual and in a population. In some embodiments the methods identify which alleles are present in a sample. In some embodiments the methods determine heterozygosity or homozygosity at one or more loci. In some embodiments, where SNPs are being interrogated for genotype, a genotype is determined for more than 75%, 85% or 90% of the SNPs interrogated by the array. In some embodiments the hybridization pattern on the array is analyzed to determine a genotype. In some embodiments analysis of the hybridization is done with a computer system and the computer system provides a determination of which alleles are present.

In one embodiment target sequences are selected from the subset of fragments that are less than 1,000 base pairs. An in silico digestion of the human genome may be used to identify fragments that are less than 1,000 base pairs when the genome is digested with the restriction enzyme, XbaI. The predicted XbaI fragments that are under 1,000 base pairs may be analyzed to identify SNPs that are present on the fragments. An array may be designed to interrogate the SNPs present on the fragments and the probes may be designed to determine which alleles of the SNP are present. A genomic sample may be isolated from an individual, digested with XbaI, adaptors are ligated to the fragments and the fragments are amplified. The amplified sample may be hybridized to the specially designed array and the hybridization pattern may be analyzed to determine which alleles of the SNPs are present in the sample from this individual.

In some embodiments the size range of fragments remains approximately constant, and the target sequences present in the size range vary with the method of fragmentation used. For example, if the target sequences are SNP containing fragments that are 400-800 base pairs, the fragments that meet these criteria when the human genome is digested with XbaI will be different than when the genome is digested with EcoRI, although there may be some overlap. By using a different fragmentation method but keeping the amplification conditions constant different collections of target sequences may be analyzed. In some embodiments an array may be designed to interrogate target sequences resulting from just one fragmentation condition and in other embodiments the array may be designed to interrogate fragments resulting from more than one fragmentation condition. For example, an array may be designed to interrogate the SNPs present on fragments that are less than 1,000 base pairs when a genome is digested with XbaI and the SNPs present on fragments that are less than 1,000 base pairs when a genome is digested with EcoRI.

In many embodiments an enzyme is selected so that digestion of the sample with the selected enzyme followed by amplification results in a sample of a complexity that may be specifically hybridized to an array under selected conditions. For example, digestion of the human genome with XbaI, EcoRI or BglII and amplification with PCR reduces complexity of the sample to approximately 2%. In another embodiment the sample is digested with an enzyme that cuts the genome at frequencies similar to XbaI, EcoRI or BglII, for example, SacI, BsrGI or BclI. Different complexity levels may be used. Useful complexities range from 0.1, 2, 5, 10 or 25% to 1, 2, 10, 25 or 50% of the complexity of the starting sample. In some embodiments the complexity of the sample is matched to the content on an array.

In many embodiments the target sequences are a subset that is representative of a larger set. For example, the target sequences may be 1,000, 5,000, 10,000 or 100,000 to 10,000, 20,000, 100,000, 1,500,000 or 3,000,000 SNPs that may be representative of a larger population of SNPs present in a population of individuals. The target sequences may be dispersed throughout a genome, including for example, sequences from each chromosome, or each arm of each chromosome. Target sequences may be representative of haplotypes or particular phenotypes or collections of phenotypes. For a description of haplotypes see, for example, Gabriel et al., Science, 296:2225-9 (2002), Daly et al. Nat Genet., 29:229-32 (2001) and Rioux et al., Nat Genet., 29:223-8 (2001), U.S. patent application Ser. No. 10/213,272 and WIPO publication number US02/25219 each of which is incorporated herein by reference in its entirety.

The methods may be combined with other methods of genome analysis and complexity reduction. Other methods of complexity reduction include, for example, AFLP, see U.S. Pat. No. 6,045,994, which is incorporated herein by reference, and arbitrarily primed-PCR (AP-PCR) see McClelland and Welsh, in PCR Primer: A laboratory Manual, (1995) eds. C. Dieffenbach and G. Dveksler, Cold Spring Harbor Lab Press, for example, at p 203, which is incorporated herein by reference in its entirety. Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/512,300, 09/916,135, 09/920,491, 09/910,292, and 10/013,598, which are incorporated herein by reference in their entireties.

In some embodiments repetitive sequences are depleted from the genomic DNA. This depletion may be done before amplification. The human genome contains various classes of repetitive DNA. The transposon derived repeats, which include short interspersed elements (SINEs), long interspersed elements (LINEs), long terminal repeat (LTR) retrotransposons, and DNA transposons, make up approximately 45% of the genome. In humans, the major family of SINEs, Alu repeats, are found ubiquitously and number approximately 1.1 million in the haploid genome. Repetitive sequences can often interfere with experimental methods which require hybridization of single-copy loci to probe sequences. For example, human Cot-1 DNA is commonly used to block non-specific hybridization in microarray experiments. In some embodiments human genomic DNA is depleted of repetitive sequences. This could improve the ability of single or low-copy sequences to specifically hybridize to probe sequences fixed to a solid support, such as beads or a microarray.

In one embodiment human Cot-1 DNA is prepared and fragmented to an average size of 50 to 300 base pairs. Alternatively, DNA oligonucleotides containing consensus sequences for different human repetitive element classes are synthesized. DNA molecules are labeled at the 3′ end with biotin-ddATP and terminal deoxynucleotidyl transferase (TdT). The biotinylated molecules are bound to streptavidin-coated microspheres (beads). Human genomic DNA is either sheared or restriction-enzyme digested, denatured, and then hybridized to beads containing repetitive elements. Multiple rounds of hybridizations can be carried out. The non-hybridized DNA, depleted of repetitive sequences, is recovered and used for further experimentation. See also, Craig et al. Hum Genet. 100(3-4):472-6 (1997).

One method that has been used to isolate a subset of a genome is to separate fragments according to size by electrophoresis in a gel matrix. The region of the gel containing fragments in the desired size range is then excised and the fragments are purified away from the gel matrix. The SNP consortium (TSC) adopted this approach in their efforts to discover single nucleotide polymorphisms (SNPs) in the human genome. See, Altshuler et al., Science 407: 513-516 (2000) and The International SNP Map Working Group, Nature 409: 928-933 (2001) both of which are herein incorporated by reference in their entireties for all purposes.

PCR amplification of a subset of fragments is an alternative, non-gel-based method to reduce the complexity of a sample. PCR amplification in general is a method of reducing the complexity of a sample by preferentially amplifying one or more sequences from a complex sample. This effect is most obvious when locus specific primers are used to amplify a single sequence from a complex sample, but it is also observed when a collection of sequences is targeted for amplification.

There are many known methods of amplifying nucleic acid sequences including e.g., PCR. See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159, 4,965,188 and 5,333,675 each of which is incorporated herein by reference in their entireties for all purposes.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double stranded DNA. Polymerases that are useful for PCR include, for example, Taq DNA polymerase, Tth DNA polymerase, Tfl DNA polymerase, Tma DNA polymerase, Tli DNA polymerase, Pfx DNA polymerase and Pfu DNA polymerase. There are many commercially available modified forms of these enzymes including: AmpliTaq® AmpliTaq® Stoffel Fragment and AmpliTaq Gold® available from Applied Biosystems (Foster City, Calif.). Many are available with or without a 3- to 5′ proofreading exonuclease activity. See, for example, Vent® and Vent® (exo-) available from New England Biolabs (Beverly, Mass.).

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554,517, and 6,063,603 each of which is incorporated herein by reference in their entireties).

When genomic DNA is digested with one or more restriction enzymes the sizes of the fragments are randomly distributed over a broad range. Following adaptor ligation, all of the fragments that have adaptors ligated to both ends will compete equally for primer binding and extension regardless of size. However, standard PCR typically results in more efficient amplification of fragments that are smaller than 2.0 kb. (See Saiki et al. Science 239, 487-491 (1988) which is hereby incorporated by reference it its entirety). The natural tendency of PCR is to amplify shorter fragments more efficiently than longer fragments. This inherent length dependence of PCR results in efficient amplification of only a subset of the starting fragments. Those fragments that are smaller than 2 kb will be more efficiently amplified than larger fragments when a standard range of conditions are used. This effect may be related to the processivity of the enzyme, which limits the yield of polymerization products over a given unit of time. The polymerase may also fail to complete extension of a given template if it falls off the template prior to completion. What is observed is that longer templates are less efficiently amplified under a standard range of PCR conditions than shorter fragments. Because of the geometric nature of PCR amplification, subtle differences in yields that occur in the initial cycles will result in significant differences in yields in later cycles. (See, PCR Primer: A Laboratory Manual, CSHL Press, Eds. Carl Dieffenbach and Gabriela Dveskler, (1995), (Dieffenbach et al.) which is herein incorporated by reference in its entirety for all purposes.) Variations in the reaction conditions such as, for example, primer concentration, extension time, salt concentration, buffer, temperature, and number of cycles may alter the size distribution of fragments to some extent. Inclusion of chain terminating nucleotides or nucleotide analogs may also alter the subset of fragments that are amplified. (See, Current Protocols in Molecular Biology, eds. Ausubel et al. (2000), which is herein incorporated by reference in its entirety for all purposes.) The presence or absence of exonuclease activity may also be used to modify the subset of fragments amplified. (See, for example, PCR Strategies, eds. Innis et al, Academic Press (1995), (Innis et al.), which is herein incorporated by reference for all purposes).

Ligation of a single adapter sequence to both ends of the fragments may also impact the efficiency of amplification of smaller fragments due to the formation of pan-handle structures between the resulting terminal repeats, see, for example, Qureshi et al. GATA 11(4): 95-101, (1994), Caetano-Anolles et al. Mol. Gen. Genet. 235: 157-165 (1992) and Jones and Winistorfer, PCR Methods Appl. 2:197-203 (1993). Smaller fragments are more likely to form the pan-handle structure and the loop may be more stable than longer loops.

In some embodiments sequences that are on smaller fragments, for example, fragments less than 400 bp or less than 200 bp are not selected as target sequences. In addition to the bias against amplification of these fragments when a single adapter is used there are also fewer small fragments following fragmentation with a restriction enzyme or enzymes. For many enzymes fragments that are, for example, smaller than 200 base pairs are relatively rare in the sample being amplified because of the infrequency of the recognition site. Since the small fragments are rare and account for relatively little sequence information there is also a decreased probability that sequences of interest will be present on small fragments.

In some embodiments the potential for formation of a stable duplex between the ends of the fragment strands is reduced. In one embodiment the adapter contains internal mismatches. In another embodiment the two strands of the adapter have a region of complementarity and a region of non-complementarity. The region of complementarity (A and A′) is near the end that will ligate to the fragments. The fragments can be amplified using primers to the non-complementary regions (B and C). Amplified products will have sequences B and C at the ends which will destabilize basepairing between A and A′. In another embodiment the sample may be amplified with a single primer for at least some of the cycles of amplification.

In some embodiments two or more different adaptors are ligated to the ends of the fragments. Ligation of different adaptor sequences to the fragments may result in some fragments that have the same adaptor ligated to both ends and some fragments that have two different adaptors ligated to each end. Small fragments that have different adaptors ligated to each end are more efficient templates for amplification than small fragments that have the same adaptor ligated to both ends because the potential for base pairing between the ends of the fragments is eliminated or reduced.

In one embodiment, the fragmented sample is fractionated prior to amplification by, for example, applying the sample to a gel exclusion column. Adaptors may be ligated to the fragments before or after fractionation. For example, to exclude the shortest fragments from the amplification the fragments can be passed over a column that selectively retains smaller fragments, for example fragments under 400 base pairs. The larger fragments may be recovered in the void volume. Because the shortest fragments in the PCR would be approximately 400 base pairs, the resulting PCR products will primarily be in a size range larger than 400 base pairs.

The materials for use in the present invention are ideally suited for the preparation of a kit suitable for obtaining an amplified collection of target sequences. Such a kit may comprise various reagents utilized in the methods, preferably in concentrated form. The reagents of this kit may comprise, but are not limited to, buffer, appropriate nucleotide triphosphates, appropriate dideoxynucleotide triphosphates, reverse transcriptases, nucleases, restriction enzymes, adaptors, ligases, DNA polymerases, primers, instructions for the use of the kit and arrays.

In order to interrogate a whole genome it is often useful to amplify and analyze one or more representative subsets of the genome. There may be more than 3,000,000 SNPs in the human genome, but tremendous amounts of information may be obtained by analysis of a subset of SNPs that is representative of the whole genome. Subsets can be defined by many characteristics of the fragments. In a preferred embodiment of the current invention, the subsets are defined by the proximity to an upstream and downstream restriction site and by the size of the fragments resulting from restriction enzyme digestion. Useful size ranges may be from 100, 200, 400, 700 or 1000 to 500, 800, 1500, 2000, 4000 or 10,000. However, larger size ranges such as 4000, 10,000 or 20,000 to 10,000, 20,000 or 500,000 base pairs may also be useful. Combinations of enzymes may be used and in some embodiments the enzymes selected cleave to generate compatible cohesive ends so that a single adaptor may be ligated to fragments resulting from digestion with 2 or more enzymes.

In one embodiment a genotyping array and assay as disclosed are used to identify regions of interest. The regions of interest may be linked to a disease of interest. The regions that are identified as being of interest by the genotyping array may then be used further analyzed. Resequencing arrays may be designed to identify novel polymorphisms in a sequence of interest and may be designed and synthesized to resequence a particular region. Resequencing arrays are available from Affymetrix, Inc. Santa Clara, Calif., for example, CustomSeq™ arrays may be designed to interrogate regions of 30 Kb or more for sequence variation. Resequencing arrays may be used to discover novel SNPs in a region of interest. Multiple method for obtaining genomic information such as genotyping a set of SNPs that are spread throughout the genome, analysis of sequence variation over a genomic region, targeted analysis of gene expression and analysis of whole genome expression are complementary approaches to obtain genomic information that may be combined and used in parallel or in series to obtain information about a biological system, for example, to obtain information about a disease. Microarrays such as those available from Affymetrix may be used in each of these methods. Large regions may be identified by linkage or association studies and those regions may be subjected to a more detailed analysis using, for example, resequencing arrays. See also U.S. Provisional Patent Application No. 60/409,396, U.S. patent application Ser. No. 10/028,482 and PCT Application No. US02/41478.

The disclosed methods may be applied to many different organisms including plants, bacteria and animals, including, human, mouse, rat, and dog. Organisms whose genomes have been sequenced are particularly useful. The genomic DNA sample may be isolated according to methods known in the art. It may be obtained from any biological or environmental source, including plant, animal (including human), bacteria, fungi or algae. Any suitable biological sample may be used for assay of genomic DNA. Convenient suitable samples include whole blood, tissue, semen, saliva, tears, urine, fecal material, sweat, buccal, skin and hair.

C. Linkage Disequilibrium Mapping Based on Ancestral Allele Identification

Linkage disequilibrium (LD) or allelic association is defined as specific alleles (i.e a and b) at two loci (A and B) that are observed together on a chromosome (haplotype ab) more often than expected from their frequencies in the population (i.e. frequency that is statistically higher than Pa x Pb that is the expected frequency if the alleles segregate independently, where Pa is the frequency of allele a and Pb is the frequency of allele b). Various methods have been proposed to measure the amount of LD and are reviewed in Jorde LB (2000, Genome Research, 10: 1435-1444). LD around an allele arises because of selection or population history. LD decays over time in any given population, due to recombination, mutation, gene conversion, natural selection, demographic structure (such as a population bottleneck), which breaks down the ancestral haplotype, i.e set of SNP alleles found along a chromosome. See also, U.S. patent application Ser. Nos. 10/316,629 and 10/316,517 which are both incorporated herein by reference in their entireties.

SNPs are the preferred tool for measuring linkage disequilibrium. Key advantages of using SNPs lie in the availability of high throughput and inexpensive typing methodologies, and higher density and mutational stability when compared with other markers such as microsatellites. Also, because microsatellites are multiallelic, the measure of LD between microsatellites is far more complex than between biallelic SNPs that occur approximately every 1 kb in the human genome.

It is believed that the complete genome sequence will reveal at least millions of SNPs. Although most SNPs are neutral or nun-functional, some are functional variants that contribute to phenotypes, such as disease susceptibility and resistance, for example. The variant itself can influence the phenotype of the gene in which it is located by affecting directly the protein or by disturbing the regulatory region of the gene leading to inappropriate levels of protein expression. Alternatively, non-functional variants SNPs can be in allelic association with a functional variant; by determining the genotype of the non-functional SNP the genotype of the functional SNP may be inferred. SNPs therefore provide a platform for identifying genomic regions directly or indirectly associated with a predisposition to a disease or another phenotype of interest.

There is a great interest in determining the patterns of linkage disequilibrium across the human genome for purposes of identifying variants associated with complex human phenotypes, e.g. common diseases such as diabetes, cardiovascular diseases, hypertension and mental illness. Initial reports sampling genome-wide LD show a wide variation of LD across the genome and across populations (Reich D. E. et al., Nature, 411:199-204, 2001). The variability of LD in genomic regions may be due to factors such as selection, local mutation and recombination rates whereas the variability of genome-wide patterns of LD is due to population factors such as population size and admixture between differentiated populations. For instance, data analysis of LD among populations reports that European populations typically display lower nucleotide diversity and greater LD than African populations, suggesting that LD mapping will be easier and more useful in European populations than in African populations (Reich, 2001; Frisse, Am. J. Hum. Genet., 69: 831-843, 2001). Biases also arise due to the fact that regions with genes that are easier to map are being studied more intensively and therefore lead to more extensive LD information for those regions. Methods that sample the entire genome without bias would be useful for determining LD in other, less studied regions in addition to relatively well studied regions.

It will also be important to estimate how many SNPs are needed to map genes from complex diseases. The extent of LD between markers has been the subject of considerable debate. Estimates of LD range extend from only a few kb (Kruglyak, Nature Genetics, 22:139-144, 1999; Dunning AM et al., Am. J. Hum. Genet., 67: 1544-1554, 2000) to over 100 kb (Abecassis et al, Am. J. Hum. Genet., 68: 191-197, 2001; Collins et al., PNAS, 96: 15173-15177,1999). In Krugylak a simulation approach was used to estimate that 500,000 markers are required to identify genetic risk factors in a random genome scan. However, this simulation is based on a regularity of behavior of LD across the genome and on a simplified model of population history. An estimate of 1,000,000 SNPs would be required when a greater variety of populations are studied (Roberts, Science, 287: 1898-1899, 2000).

Alternatively, SNPs can be grouped in bins/blocks as distinct haplotypes and fewer haplotype SNPs would be required to map disease phenotypes. Therefore extended linkage disequilibrium can be detected across a large region including several haplotype blocks using a few SNPs. Daly and colleagues show a highly structured pattern of LD with regions or blocks of high LD separated with hot spots of recombination with a breakdown of LD (Daly M. J. et al., Nature Genet., 29: 233-237, 2001). Patil et al. report that using high density oligonucleotide arrays to human chromosome 21 reveals only three common haplotype variations among 80% of the human population (Science, 294: 1719, 2001). Haplotype blocks can be composed from haploid (see U.S Patent application 2003009964 A1, incorporated herein by reference in its entirety) or diploid data. At a given polymorphic site, any diploid individual can be either homozygous (two copies of the same allele) or heterozygous (two different alleles) defining more than one haplotype. This complexity can be overcome by statistical estimation of haplotype frequencies using algorithms such as the expectation-maximization (EM) algorithm and Clark's algorithm (Clark, 1990, Mol Biol Evol, 7:111-122). With these blocks defined, the variation in the human genome could be represented by a relatively small fraction of SNPs.

Reich and colleagues showed that in some genomic regions in some populations an appreciable extent of linkage disequilibrium was found between markers separated by up to 60 kb. In other populations the same study found linkage disequilibrium in another population extending only to regions separated by about 5 kb. Different populations will have different average extents of LD and in some embodiments ancestral allele analysis may be used to estimate extent of linkage in different regions to determine average extent of LD in a given population.

In another embodiment variance of LD as a function of distance between sites may also be estimated. If variance of LD is predicted to be low all sites separated by a given distance, for example 10 kb, are predicted to almost always be in strong LD while sites farther than, for example 20 kb almost never are. With this scenario a single marker may be sufficient to be indicative of a 10 kb interval. If variance of LD is predicted to be high then a single marker would not be sufficient to mark off a 10 kb interval. See, Goldstein and Weale, Current Biology 11:R576-R579 (2001). In one embodiment determination of ancestral alleles and the frequency of ancestral and non-ancestral alleles is used to estimate variance of LD.

SNPs may be present in a polymorphic form in one population and not polymorphic in a second population. SNP alleles may also be present at different frequencies in different populations. For example, the non-ancestral allele in one population may be present at 90% and the non-ancestral allele at 10% frequency while the same SNP in another population may have 90% non-ancestral allele and 10% ancestral allele. Typically the frequency of the less common allele of the biallelic markers of the present invention is greater than 1%, preferably the frequency is greater than 10%, more preferably the frequency is at least 20%, even more preferably the frequency is at least 30%.

The extent of LD decreases in proportion to the number of generations since the LD-generating event and varies with the population history. Methods of predicting the extent of LD in a given genomic region or population are disclosed. The highly variable average extent of linkage disequilibrium highlights the need to adjust the marker densities by region and to come up with a global map of LD in the human population. Such a map will allow the researcher to select the number and type of SNPs that will work best for a specific region. Ancestral alleles may be used to generate a map of LD based on the prediction that a recently appearing SNP allele is likely associated with a greater length of shared DNA than an ancient allele. See, Collins et al., Science 278:1580-1581 (1997), Kruglyak, Nature Genet. 17:21-24 (1997), Nickerson et al., Nature Genet. 19:233-240 (1998) and Lai et al., Genomics 54:31-38 (1998) each of which is incorporated herein by reference.

D. Method

1. Determination of Ancestral Allele

Human and chimpanzees shared a common ancestor 5-6 million years ago. Gorillas and humans shared a common ancestor 8 millions years ago. Chimpanzee and gorilla nucleotide sequence of the genomic DNA differ from human only by 1.5% and 2%, respectively, falling into the normal range of DNA variation between different human individuals (Hacia J. G., Trends Genet., 17:637-45, 2001). Studies using high-density oligonucleotide arrays suggest that genomic rearrangement provides the genetic basis for the qualitative and quantitative differential gene expression between human and non-human primates. Comparison of human chromosome 21 with non-human primate DNA sequences using high density oligonucleotide arrays identified a significant number of random genomic rearrangements that have occurred frequently during primate genome evolution. These DNA rearrangements are commonly found in segments containing genes, suggesting possible functional consequences (Frazer K et al., Genome Research, 13: 341-346, 2003).

Human biallelic SNPs comprise two alleles, one that is likely to be ancestral and one that likely arose more recently. The site may also be polymorphic in chimpanzees or apes, sharing the same or different alleles. If chimpanzee and gorilla DNA is not polymorphic at a given human SNP site, one of the two human alleles is usually present as a homozygote in great apes. Using oligonucleotides microarrays, Hacia and colleagues have determined ancestral alleles for human SNPs by identifying SNPs that give the same homozygous specification in chimps and gorillas (Hacia et al., Nature Genetics, 22:164-167, 1999). In one embodiment of the present invention ancestral alleles are determined for more than 1,000, more than 5,000 or more than 10,000 human SNPs distributed throughout the human genome using a generic amplification method that does not require the use of locus specific primers. Although the method does not use locus specific amplification the genomic regions that are amplified are amplified reproducibly depending on the enzyme or enzymes used to digest the genome before amplification and the amplification reaction conditions used. In many embodiments genotyping is done using an array that is designed to determine which allele or alleles are present at the SNPs that are known to be reproducibly amplified by the disclosed amplification method.

In one embodiment the present invention estimates the extent of DNA that is in linkage disequilibrium with a human SNP by identifying the ancestral allele using whole genome analysis arrays. The older allele is assumed to be the common allele between humans and chimpanzees, or gorilla. For example, if the SNP in humans is AB, a BB genotype in chimpanzee and gorilla at that position is an indication that the B allele is the older allele. The identification of the ancestral nucleotide is an indication of the relative age of a polymorphism and enables the determination of which polymorphism appeared more recently in the human population examined. In one embodiment, the information about age allows the identification of regions of the genome that are likely to have lower levels of LD near the older allele, due to a decay over time. In another embodiment the method allows haplotype reconstitution i.e. the establishment of the alleles of a set of SNPs that are inherited together on a single chromosome. Estimates of haplotype frequencies can be calculated through statistical algorithm such as the EM algorithm (Dempster A. P. et al., 1977, J. Royal Stat. Soc. B, 39: 1-38). Haplotypes are valuable for medical genetic studies because they allow mapping of disease susceptibility alleles without the need to discover and test every SNP across a chromosomal region.

For assay of genomic DNA, virtually any biological sample (other than pure red blood cells) is suitable. For example, convenient tissue sample include whole blood, saliva, buccal, tears, semen, urine, sweat, fecal material, skin and hair. Sample preparation, hybridization and data analysis are provided in various sections of this specification and cited references. Methods of genotyping using a nucleic acid array following complexity reduction have been described, for example, in U.S. Pat. Nos. 6,361,947, 6,391,592 and U.S. patent application Ser. Nos. 09/916,135, 09/920,491, 09/910,292, 10/264,945 and 0036069-A1 and in Dong S. et al. (2001), Genome Res., 11:1418-1424, each of which are incorporated herein by reference in their entireties. In brief, this method comprises fragmenting nucleic acid samples to form fragments, ligating adaptors to the fragments and amplifying the fragments under conditions that favor amplification of fragments of a particular size. (For example see U.S. Pat. No. 6,509,160, which is incorporated by reference in its entirety for all purposes). In silico digestion is used in many embodiments to predict the SNPs that will be present on each fragment when a genome is digested with a particular restriction enzyme or enzymes. The SNPs and corresponding fragment sizes can be further separated by computer into subsets according to fragment size. The information is then used to design arrays to interrogate SNPs predicted to be present in a particular size fraction resulting from a particular digestion and amplification method. Arrays may be designed to interrogate a particular subset of sequences or fragments that may include a subset of polymorphisms. In some embodiments a subset of a genome is isolated by, for example, preferential amplification of a subset of fragments. In some embodiments the subset of fragments that is preferentially amplified are those fragments that are of a selected size range, for example, between 1, 200, 400, 500, or 1,000 and 800, 1,000, 2,000 or 5,000 bases. During amplification some fragments will be amplified more efficiently than other fragments and those fragments that are efficiently amplified are preferentially amplified. In the final amplified product those fragments that are preferentially amplified will be present at relatively high levels compared to fragments that are inefficiently amplified. Fragments that are preferentially amplified are present in the amplified product at levels that are high enough so that there is enough to hybridize to a genotyping array and make an accurate call of the genotype of the SNP or SNPs present on that fragment.

In many embodiments a reproducible, high throughput method of amplifying a representative subset of the human genome is provided. In brief, genomic DNA is digested with one or several restriction enzymes and ligated to an adaptor that has an overhang that is complementary to the overhang generated by the restriction enzyme or enzymes, if the enzyme generates a blunt end without an overhang the adaptor may have no overhang. All fragments resulting from enzyme digestion, regardless of size, are substrates for adaptor ligation. A generic primer that recognizes the adaptor sequence, is used to amplify ligated DNA fragments. In one embodiment 200-500 ng of DNA is used for each sample to be genotyped, in one embodiment about 250 ng is used. The DNA may be digested in a single tube and ligated to the adapter in the same tube. The fragmented, ligated DNA may be amplified in 1, 2, 4, 6 or 8 separate PCR reactions which are combined at a later step prior to fragmentation, labeling and hybridization to a genotyping array, see, for example, GeneChip® Mapping Assay Manual (2003) available from Affymetrix, Inc., Santa Clara, Calif. which is incorporated herein by reference in its entirety.

In another embodiment a high throughput genotyping method is disclosed, the method is capable of genotyping more than 5,000, 10,000, 50,000, 100,000 or 500,000 different SNPs in each of 96 or more samples simultaneously. In this embodiment each step is performed in a 96 well plate. In some embodiments appropriate volumes of reaction components are added by a robot. The plates may be handled by a robot and a bar code system may be used to track the samples, for additional methods of high throughput analysis see WO application number US02/41478 and U.S. application Ser. No. 10/028,482 which are incorporated herein by reference in their entireties.

In many embodiments the array is designed to interrogate one or more polymorphic positions predicted to be present in the fragments of the subset of fragments isolated or amplified. The amplification method used results in reproducible amplification of a known set of SNP containing fragments. In many embodiments an array is designed to genotype the set of SNPs that is reproducibly amplified given the fragmentation and amplification conditions, see U.S. patent application Ser. No. 10/264,945 and U.S. Provisional Patent Application Nos. 60/417,190 and 60/470,475 each of which is incorporated herein by reference.

In some embodiments SNPs are amplified by target specific amplification using one or more primers that hybridize near the SNP or interest. A collection of SNPs may be amplified using a collection of target specific primers. The array may be designed to interrogate the SNPs in the collection of SNPs, see, for example, U.S. patent application Ser. No. 10/272,155.

2. Relationship Between Human SNP Allele Frequencies and their Ancestral State.

The allele frequency is the frequency with which a selected allelic form (for example SNP allelic form) of a gene exists in a population or selected group of organisms. Evolution occurs when allele frequencies change in a population: Allele frequency close to 0 may indicate loss of allele whereas allele frequency close to 1 may indicate allele fixation. The allele frequency spectrum in a given population bears the signatures of its history. Forces such as natural selection, random genetic drift, demographic events such as population bottlenecks or expansions, or some combination of all of these, manifest their effects on populations. Demographic events and genetic drift are expected to have genome-wide effects on the allele frequency spectrum, while natural selection exerts its effects on a few specific loci in the genome.

The frequency of the selected allelic form can be quantified as the detected number of copies of the selected allele divided by the total number of alleles of the gene possessed by the individuals tested. (For example, see U.S. Pat. No. 6,368,799, which is incorporated by reference in its entirety for all purposes). Statistical methods are available to determine whether the number of individuals tested is representative of a given population. In one embodiment, allele frequency is determined from the relative intensity of hybridization to probes. The ratio of different allelic forms in a population is determined by measuring the ratio of the relative intensities of the label which hybridizes to the probes corresponding to the different allelic forms. When two SNPs are in linkage disequilibrium change in the frequency of one allele at one SNP results in a similar change in the frequencies of alleles at the other SNP. Also, the extent of association among alleles may differ depending on the frequencies of the alleles. In one embodiment, extent of LD is predicted by examining the relationship (i.e. the correlation coefficient) between the ancestral allele state and the SNP allele frequency in ethnically distinct human populations. In one embodiment, allele frequency and ancestral allele state are in strong correlation for specific populations and allele frequency is used to determine the extent of linkage disequilibrium. In one embodiment, correlation coefficient is greater than 0.7. In a preferred embodiment, correlation coefficient is greater than 0.8.

3. Discovery of Ancestry-Informative Markers (AIMs)

In one embodiment the F_{ST} statistic is calculated and used to identify SNPs that are ancestry-informative markers (AIMs), F_{ST} is an estimate of the geographic structure between two populations, for each SNP. F_{ST} values vary from 0 to 1; as allele frequency differences between populations become more pronounced, F_{ST} values increase. When calculating 0.061, 0.094 and 0.065 for SNPs in an African-American versus Caucasian population, African-American versus Asian populations and Caucasian versus Asian populations the mean F_{ST} values are typically less than 0.1 indicating that the majority of markers show very small inter-population frequency differences. However, there is a subset of SNPs whose allele frequencies differ significantly in one population versus the other two. These SNPs, called ancestry-informative markers, or AIMs, can be used to map complex diseases using admixture-generated linkage disequilibrium, or MALD. See Collins-Schramm, H. et al., Am. J. Hum. Genet. 70, 737-750 (2002), Briscoe, D. et al., J. Hered. 85, 59-63 (1994), Parra, E. J. et al., Am. J. Hum. Genet. 63, 1839-1851 (1998), and McKeigue, P. M. et al., Ann. Hum. Genet. 64, 171-186 (2000) each of which is incorporated herein by reference in its entirety.

### E. EXAMPLES

### Example 1

### Ancestral allele determination

SNPs are “mutations” that have arisen during evolution. To establish which of the two alleles represents the ancestral state, genotypes were determined in chimpanzee and gorilla genomic DNA samples at positions corresponding to polymorphic positions in humans. Genotypes were determined using high density oligonucleotides arrays designed to genotype a plurality of humans SNPs, such arrays are available from Affymetrix, Inc., Santa Clara, Calif. and are described, for example, in U.S. Provisional Patent Application Nos. 60/470,475 and 60/417,190. Methods of genotyping using these arrays are described in, for example, U.S. patent application Ser. No. 10/264,945 and in the GeneChip® Mapping Assay Manual (2003). The allele that is present in both apes is considered to be the more ancient of the two alleles in humans. Additional methods of using these arrays are also described in, for example, U.S. Provisional Patent Application Nos. 60/319,685, 60/319,253 and 60/468,925.

Genotypes for 11,863 human SNP sites were scored in all animals. Genotypes were called as homozygous for either allele A or B (AA or BB), heterozygous (AB) or undetermined (no call). Summary results for genotypes in chimp and gorilla are shown in Table 1. Absolute numbers of genotype calls and their percentages are shown. The results indicate that chimpanzee and gorilla genotypes can be called on 76% and 70% of the human SNPs, respectively. Call rates for the same genotypes in humans were approximately 93%. As expected the call rate was lower in chimpanzees and gorilla due to genomic sequence variation. Variation between species in the sequence surrounding the SNPs may result in lower hybridization and poorer call rates in the non-human species because the probes of the array were designed based on the human sequence.

TABLE 1

| | Human | Chimpanzee | Gorilla |
|---|---|---|---|
| Number A Calls | 3473 | 4244 | 3854 |
| Number B Calls | 3381 | 4342 | 4010 |
| Number AB Calls | 3924 | 218 | 248 |
| Number No Calls | 774 | 2748 | 3440 |
| Number Total Calls | 10778 | 8804 | 8112 |
| Number Calls | 11552 | 11552 | 11552 |
| Attempted | | | |
| % Call Rate | 93.3 | 76.2 | 70.2 |
| % A | 32.2 | 48.2 | 47.5 |
| % B | 31.4 | 49.3 | 49.4 |
| % AB | 36.4 | 2.5 | 3.1 |

The overwhelming majority of markers are homozygous in both great ape species (Table 1), consistent with the recent evolutionary history of SNPs. Of the 11,552 SNPs analyzed 24 SNPs were identified that were heterozygous in both humans and chimpanzees, and 34 SNPs were identified that were heterozygous for all three species. This result suggests one of two possibilities. The first possibility is that these SNPs are extremely ancient, occurring more than 5 million years ago and may represent shared polymorphisms. This would predict a very limited extent of LD near this particular SNP. The second possibility is that the polymorphism was caused by a repeated mutation at the same position. These long-lived polymorphisms are believed to favor diversity such in the case of alleles of the class I and class II major histocompatibility complex genes. The latter possibility suggests that this polymorphism is not neutral because it is unlikely that a neutral gene could retain such a variation since the time of the common ancestor. Most human neutral polymorphism is indeed expected to be less than 800,000 years old.

Ancestral alleles were assigned only to SNPs that met the following criteria: SNPs that were homozygous in both chimpanzee and gorilla giving the same genotype call in both species and that shared one allele with humans. A total of 6925 SNPs were assigned. By identifying ancestral nucleotides, one can determine which polymorphism appeared in the human population most recently.

### Example 2

### Determination of the correlation between the SNP frequencies and the ancestral allele state in three different populations

Consistent with theoretical predictions (Watterson G. A. and Guess H. A., Theor Popul. Bio.l,11: 141-60, 1977), previous results for 214 SNPs in an ethnically diverse set of samples showed that the most frequent allele is not always ancestral (Hacia, 1999). In this example the relationship between frequency and ancestral allele was examined in three human populations. Allele frequencies were determined for 13,647 SNPs in DNA from 60 unrelated individuals comprising three human populations: African-American, Caucasian and Asian. A comparison of the allele frequencies derived from a set of 20 Caucasians versus a set of 38 Caucasians showed a high correlation (correlation coefficient=0.98), indicating that sampling of 20 individuals provides reasonably stable estimates of allele frequencies for these SNPs in that population. The allele frequencies obtained were in good agreement with frequency data available from TSC as part of the allele frequency project (AFP). Data was available for 313 of the SNPs.

Of the 13,647 SNPs interrogated, the vast majority were polymorphic in all three populations. This is consistent with expectations, as the training set consisted of an ethnically diverse panel of individuals. In this analysis, there were 343, 535 and 1219 markers in the African-American, Caucasian and Asian samples, respectively, which were monomorphic (i.e. zero heterozygosity). Of these, 100 were monomorphic in both African-Americans and Asians (but not Caucasians), 81 were monomorphic in African-Americans and Caucasians (but not in Asians) and 236 were monomorphic in both Asians and Caucasians (but not African-Americans). A subset of SNPs showing extreme differences in allele frequency amongst the three populations was examined more closely, and the findings were correlated with ancestral allele information and estimates of departures from neutrality.

The distribution of the chimpanzee and gorilla (i.e. ancestral) alleles was plotted as a function of SNP allele frequency in the African-American, Caucasian and Asian populations and correlation was established in each case. The slopes of the Caucasian and Asian populations are 0.58 and 0.49, respectively. These data indicate that in these two populations the ancestral allele is not always the most frequent allele; i.e. in about 20% of the SNPs, the newer allele has become more frequent in these populations, consistent with previous studies (Watterson and Guess, 1977; Hacia, 1999). The new “mutations” i.e. non-ancestral alleles, could have reached high frequencies (i.e. near-fixation) in the non-African populations by random genetic drift, population bottlenecks, expansions or natural selection. In contrast, the slope of the curve in African-Americans is 0.95, indicating a nearly one-to-one correlation between ancestral state and allele frequency. In this population, regardless of relative allele frequency, the most frequent allele is almost always the ancestral allele, contrary to theoretical predictions (Watterson & Guess, 1977).

### Example 3

### Determination of the Correlation Between the Ancestral Allele State and the Genetic Differences Between Populations

The F_{ST} statistic (Weir, 1996 Genetic Data Analysis II. Sinauer Associates, Sunderland, Mass.), which is an estimate of the geographic structure between two populations, was calculated for each SNP using the formula below:

F_{ST}=S²/pq

Where s² is the sample variance (wherein s is the standard deviation), p is the frequency of allele A (i.e. ancestral allele) and q is the frequency of allele B (i.e. “new allele”). Then the percentage of SNPs corresponding to the non-ancestral allele was determined. F_{ST} values vary from 0 to 1: as allele frequency differences between populations become more pronounced, F_{ST} values increase. To determine whether the set of high frequency, non ancestral alleles were more or less likely to have high F_{ST} values, i.e. to show genetic differences in different populations SNPs with allele frequencies greater than 0.8 were identified in each population. F_{ST} values were determined for each pair wise population comparison and then the percentage of SNPs corresponding to the non-ancestral allele were determined. A striking positive correlation was observed between the non-ancestral state and high F_{ST} values in the Caucasian (correlation coefficient=0.95) and Asian (correlation coefficient=0.96) high-frequency SNPs. This result indicates that, in these populations, there is a direct correlation between the “new” allele and the likelihood that this SNP shows large differences between populations. In contrast, high frequency SNPs in African-Americans showed no such correlation with F_{ST} values as a function of non-ancestral state (correlation coefficient<0.34). This supports a common demographic event that occurred as populations migrated out of Africa into Eurasia. Perhaps those new SNPs rose in frequency in the Asian and Caucasian populations because they conferred some adaptive advantages to people in their new environments as they migrated out of Africa. In another embodiment SNPs with F_{ST} values greater than 0.4 or greater than 0.3 when two populations are compared are identified.

### CONCLUSION

From the foregoing it can be seen that a flexible and scalable method for analyzing genotype in complex samples of genomic DNA is disclosed. The methods provide powerful tool for analysis of complex nucleic acid samples, including methods of determining ancestral alleles for large numbers of human SNPs and for using ancestral allele information to identify regions of high or low linkage disequilibrium throughout the genome. The methods provide for fast, efficient and inexpensive analysis of complex nucleic acid samples. The methods are particularly well suited to high throughput methods of genotyping and genotyping analysis.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A method for identifying the ancestral allele of a human single nucleotide polymorphism (SNP) comprising the steps of:
a. providing a nucleic acid array comprising allele specific probes to at least 5,000 human SNPs;
b. providing a first genomic DNA from a first higher primate species and a second genomic DNA from a second higher primate species;
c. amplifying the first and second genomic DNAs using a single primer to generate a first and second amplification product;
d. generating a first and second hybridization pattern by hybridizing the first amplification product to a first copy of the nucleic acid array and the second amplification product to a second copy of the nucleic acid array;
e. analyzing the first and second hybridization patterns to identify at least one human SNP that is homozygous for the same allele in both the first and the second higher primate species; and
f. assigning the allele as the ancestral allele state of that human SNP.

2. A method for estimating extent of linkage disequilibrium in the chromosomal region near at least one human SNP comprising:
a. determining the ancestral allele for a plurality of human SNPs according to the method of claim 1;
b. identifying at least one human SNP allele that is the ancestral allele; and,
c. predicting low linkage disequilibrium across the chromosomal region near the at least one human SNP allele that is the ancestral allele.

3. A method for estimating extent of linkage disequilibrium in the chromosomal region near at least one human SNP comprising:
a. determining the ancestral allele for a plurality of human SNPs according to the method of claim 1;
b. identifying at least one human SNP allele that is the non-ancestral allele; and,
c. predicting high linkage disequilibrium across the chromosomal region near the at least one human SNP allele that is the non-ancestral allele.

4. A method according to claim 1 wherein one of the higher primate species is a chimpanzee.

5. A method according to claim 1 wherein one of the higher primate species is a gorilla.

6. A method according to claim 1 wherein one of the higher primate species is an orangutan.

7. A method according to claim 3 wherein the chromosomal region is the region that is within 1 kb of the non-ancestral allele in either direction.

8. A method according to claim 3 wherein the chromosomal region is the region that is within 100 kb of the non-ancestral allele in either direction.

9. A method according to claim 3 wherein the chromosomal region is the region that is within 200 kb of the non-ancestral allele in either direction.

10. A method of identifying at least one non-ancestral allele of a human SNP comprising:
a. identifying the ancestral allele of the SNP according to the method of claim 1; and
b. assigning any allele of the SNP that is not the ancestral allele as a non-ancestral allele.

11. A method for estimating extent of linkage disequilibrium in the chromosomal region near a plurality of human SNPs comprising:
a. determining the ancestral allele for a plurality of human SNPs according to the method of claim 1;
b. identifying the ancestral and non-ancestral alleles for each human SNP; and,
c. predicting low linkage disequilibrium across the chromosomal region near each ancestral allele and high linkage disequilibrium across the chromosomal region near each non-ancestral allele.

12. A method of establishing a pattern of regions of high linkage disequilibrium across a chromosome, the method comprising:
a. identifying a plurality of SNPs with a non-ancestral frequency greater than 0.3 in a population of individuals wherein the SNPs are found on the same chromosome;
b. predicting regions of high linkage disequilibrium near the non-ancestral alleles; and
c. establishing a pattern of regions of high linkage disequilibrium across the chromosome.

13. A method of establishing a pattern of regions of high linkage disequilibrium across a plurality of chromosomes comprising establishing a pattern of regions of high linkage disequilibrium across one chromosome according to claim 11 and repeating the method of claim 11 for at least one other SNP located on a second chromosome that is different from the first chromosome.

14. A method to establish a pattern of linkage disequilibrium across a plurality of human chromosomes comprising estimating the extent of linkage disequilibrium in the chromosomal region near at least one human SNP located on a first chromosome according to the method of claim 2 or 3 and repeating the method of claim 2 or 3 for at least one other SNP located on a second chromosome that is different from the first chromosome.

15. A method of establishing a linkage disequilibrium map across human chromosomes, the method comprising:
a. identifying at least one non-ancestral allele of a first human SNP according to claim 1,
b. identifying chromosomal regions localized less than 200 kb from the at least one non-ancestral allele;
c. identifying at least one other human SNP within this region;
d. grouping the first SNP and the SNP or SNPs identified in (c) into blocks; and,
e. predicting high linkage disequilibrium between SNPs within these blocks.

16. A method according to claim 12 or 13, further establishing a haplotype map.

17. A method of establishing a haplotype map across a human chromosome, the method comprising the steps of:
a. identifying human SNPs that are predicted to be in high linkage disequilibrium according to claim 3;
b. grouping said SNPs into blocks;
c. estimating haplotype diversity within these blocks using a computer and computer code that estimates haplotype diversity; and
d. establishing a haplotype map.

18. A method of establishing a haplotype map across a human chromosome, the method comprising the steps of:
a. identifying human SNPs that are not ancestral;
b. identifying chromosomal regions localized less than 200 kb from the non-ancestral allele;
c. identifying human SNPs within these regions;
d. grouping said SNPs into blocks of high linkage disequilibrium;
e. estimating haplotype diversity within these blocks via a haplotype estimation software; and,
f. establishing a haplotype map.

19. A method according to claim 16, wherein the haplotype map is used to search for complex disease genes.

20. A method for estimating the extent of linkage disequilibrium in the human genome, the method comprising the steps of:
a. determining the allele frequencies for a first plurality of SNPs in a population;
b. determining the ancestral allele for a second plurality of SNPs contained in said first plurality of SNPs according to the method of claim 1;
c. comparing the allele frequency of a SNP to the frequency of the ancestral allele for that SNP for a plurality of SNPs from said second plurality of SNPs in the population sample to generate a correlation coefficient for the population;
d. determining that an ancestral allele is a high frequency ancestral allele if the correlation coefficient is greater than 0.8;
e. identifying a chromosomal region nearby a high frequency ancestral allele; and,
f. inferring low linkage disequilibrium across said region.

21. A method according to claim 20 wherein the chromosomal region is localized less than 1 kb from the frequent allele.

22. A method according to claim 20 wherein the chromosomal region is localized less than 100 kb from the frequent allele.

23. A method according to claim 20 wherein the chromosomal region is localized less than 200 kb from the frequent allele.

24. A method according to claim 20 further comparing linkage disequilibrium extent among geographically distinct human populations.

25. A method according to claim 20 further comparing linkage disequilibrium extent among ethnically distinct populations.

26. A method according to claim 24 or 25, further predicting which population is more ancient.

27. A method to identify at least one ancestry informative marker comprising:
a. determining the allele frequency for each of a plurality of SNPs in each of two populations;
b. calculating an F_{ST} value for at least one SNP in said plurality of SNPs; and
c. identifying at least one SNP whose F_{ST} value is greater than 0.3.

28. The method of claim 27 further comprising identifying at least one SNP whose F_{ST} value is greater than 0.4.

29. The method of claim 27 wherein allele frequency is determined by genotyping a SNP in a plurality of individuals that are members of a population wherein genotypes are determined by hybridizing a sample from each individual to a nucleic acid array comprising allele specific probes to at least 5,000 human SNPs.

30. A method of identifying a haplotype in a region of high linkage disequilibrium in a first population of individuals, wherein a haplotype comprises at least two linked SNP alleles, comprising;
a. identifying a non-ancestral allele of a first SNP in the first population wherein the non-ancestral allele is not present in a second population and wherein the ancestral allele is determined by the method of claim 1;
b. genotyping at least one additional SNP that is within 100 kb of the first SNP; and
c. determining which allele of said additional SNP is linked to said non-ancestral allele in said first SNP.
